(19) United States
(12) Patent Application Publication
(10) Pub. No.: US 2013/0066199 A1
(43) Pub. Date: Mar. 14, 2013

(54) TUMOR MARGIN DETECTION METHOD BASED ON NUCLEAR MORPHOMETRY AND TISSUE TOPOLOGY

(71) Applicant: V Krishnan Ramanujan, Sherman Oaks, CA (US)

(72) Inventor: V Krishnan Ramanujan, Sherman Oaks, CA (US)

(73) Assignee: CEDARS-SINAI MEDICAL CENTER, Los Angeles, CA (US)

(21) Appl. No.: 13/639,180

(22) PCT Filed: Apr. 15, 2011
(86) PCT No.: PCT/US11/32707

§ 371 Date: Oct. 3, 2012

Related U.S. Application Data
(60) Provisional application No. 61/324,661, filed on Apr. 15, 2010.

(51) Int. Cl. A61B 6/00; G01N 21/47; C12M 1/00; A61K 49/00; C12Q 1/04; G01N 21/64

(52) U.S. Cl. 600/431; 435/34; 435/307.1; 424/9.6; 600/407; 356/446

(57) ABSTRACT

Systems and methods for detecting tumor margins are disclosed. The detection can be performed intra-operatively. A device is provided for housing a tissue sample during optical analysis for detection of tumor margins.

### CROSS-REFERENCE TO RELATED APPLICATIONS

This application claims priority from U.S. patent application Ser. No. 61/324,661, filed Apr. 15, 2010, the contents of which are herein incorporated by reference.

### GOVERNMENT RIGHTS

The invention was made with government support under Grant No. R21 CA124843 awarded by the National Institutes of Health. The government has certain rights to the invention.

### FIELD OF INVENTION

This invention relates to systems and methods for the detection of tumors and tumor margins.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Breast carcinoma is the most frequently diagnosed malignancy in women. Currently, a woman living in the US has a 12.3% lifetime risk of developing breast cancer. In the last two decades, the incidence rate of small (<2 cm) tumors has increased by ˜2% per year suggesting the critical role of mammography and other screening strategies in detecting early cancers. Despite this good news, breast cancer continues to account for more than 21% of cancer related deaths worldwide and for the estimated 40,000 breast-cancer related deaths in the US alone in 2010. A combination of breast-conservation surgery (lumpectomy) and radiation therapy has become a standard of treatment for most in-situ and invasive cancers [1-9]. Removing all tumors present, with ‘clear margins’, is the goal of breast-conserving surgery. Failure to do so significantly increases the risk of local recurrence. While local recurrence may be treatable (mastectomy, chemotherapy+/− radiation), it increases the risk of systemic recurrence and death.

Margin assessment depends on histopathologic analysis of the lumpectomy specimen, which typically takes 2-3 days [10-14]. Information from this analysis is thus of no immediate value during surgery. Several other approaches (e.g., imprint cytology, tomography etc.,) have shown promise but none have yet made the jump from clinical research to clinical acceptance [10, 12, 15-17]. The use of intra-operative frozen section has the longest track record. Frozen section is not as reliable as permanent (H&E) section and specimens processed in this manner cannot be evaluated further. This emphasizes the value of developing technologies that can incrementally add to the ability to detect cancer intraoperatively, even if these technologies do not have outstanding sensitivity and/or specificity. It is evident that alternate detection technologies are needed that can augment the existing repertoire of clinical diagnostic modalities. A long-term goal is to develop optical imaging approaches for enabling the tumor margin detection in intraoperative settings [18-20].

Sentinel lymph node (SLN) is the first node in the receiving basin of lymph nodes to which lymphatic drainage from an organ occurs. Axillary staging is an essential prognostic indicator for patients with invasive breast carcinoma [36]. SLN biopsy represents a minimally invasive approach to the surgical management of the axilla for patients with invasive breast cancer. In situations where SLN biopsy is not a viable option, a surgical intervention (lumpectomy and/or radiotherapy) becomes necessary [37, 38]. This increases the discomfort and morbidity for patients as well as logistic issues in clinical management of breast cancer. Our long term goal is to develop and implement high sensitive optical imaging modalities for non-invasive detection of cancer-specific signatures [39]. The rationale behind this goal is that changes in physiological status and the onset of disease pathology such as cancer would alter the optical properties of mammalian tissues thereby offering a possible avenue for their detection [20, 40, 41, 42]. With this motivation, the inventors tested the hypothesis that multispectral reflectance imaging can provide a reliable, non-invasive imaging platform for detecting tumor specific signatures in a preclinical invasive carcinoma in a rat model.

### SUMMARY OF THE INVENTION

The invention is directed to methods for detecting tumor margins in subjects in need thereof. The method comprises providing a tissue sample from the subject and measuring nuclear morphometric and/or tissue topology parameters. The nuclear morphometric and/or tissue topology parameters from the area of interest in the tissue sample are compared to the areas surrounding the area of interest in the tissue sample. A difference in the nuclear morphometric and/or tissue topology parameters between the area of interest and the surrounding tissue is indicative of a tumor margin.

The invention further provides methods for detecting a tumor in a subject in need thereof. The method comprises obtaining multispectral reflectance images in a subject, at various wavelengths, of an area of interest and of the surrounding area. The reflectance spectra thus obtained of the area of interest is compared with the reflectance spectra of the surrounding area. A difference between the reflectance spectra of the area of interest and the reflectance spectra of the surrounding area is indicative of the presence of a tumor.

The invention also provides an apparatus to support a tissue sample during data acquisition, comprising a scaffold configured to enclose the tissue sample and a mechanism to support the scaffold, adapted to position the tissue sample for optical analysis

### BRIEF DESCRIPTION OF THE FIGURES

Exemplary embodiments are illustrated in referenced figures. It is intended that the embodiments and figures disclosed herein are to be considered illustrative rather than restrictive.

FIG. 1: Nuclear Morphometry/Topology Analysis Schematic: In connection with an embodiment of the invention, a two-dimensional image of fluorescent microbeads of various sizes is shown (a). This situation mimics the nuclear distribution in a typical tissue labeled with the intercalating dye, DAPI. Image segmentation process begins with intensity thresholding of the raw image (b). This step addresses the heterogeneity in fluorescence intensity across the field-of-view. The next step is to render the thresholded binary image to detailed morphometric analysis by either of the two methods: edge detection (c) or watershed algorithm (d). Morphometric parameters of relevance to this study are (i) nuclear size, (ii) nuclear circularity and (iii) nuclear area fraction as defined in the text and exemplified in (e). In complex images where the nuclear area fraction is high, the above two image segmentation approaches can yield an underestimate of the calculated nuclear volume fractions. This situation occurs when the overlap of neighboring nuclei (e.g., tumor regions) exceeds the optical resolution of the imaging system (˜0.25 μm). In order to address this inherent limitation, the processed images are also analyzed for topological information such as connectedness and fractal dimension. Together, morphometric and topological analyses of the tissue fluorescence images provide a comprehensive picture of the tissue architecture.

FIG. 2: Nuclear Morphometry/Topology Analysis in Thin sections of Breast Tumor Tissues: In connection with an embodiment of the invention, representative nuclear fluorescence image of a tumor tissue section with a bordering normal epithelium is shown (a) (Scale bar=200 μm). The nuclear area fraction is significantly higher in the tumor region as compared with that of the normal epithelium. In order to quantify these differences, morphometric parameters were analyzed in multiple tissue sections and presented here. Image segmentation by watershed algorithm (b) and edge-detection algorithm (c) yielded two different models for quantifying the nuclear distribution in the images. The original image (915 μm×684 μm) was divided into regular subunits of size (20 μm×684 μm). Mean nuclear count in each image subunit by the two aforementioned algorithms as shown in FIG. (d). Although both the algorithms yielded similar spatial profile of nuclear distribution in the tissue images, the edge-detection approach was found to be more accurate in delineating the individual nuclei in a cluster whose size was beyond the resolution of the optical imaging system. Fractal dimension was also computed in these image subunits as described in the main text and presented in FIG. (e). Mean nuclear size and circularity are shown in FIGS. (f) and (g).

FIG. 3: Statistical Analysis of Nuclear Morphometry Parameters in Breast Tissues: In connection with an embodiment of the invention, nuclear morphometry parameters were calculated in multiple images of normal and breast-tumor specimens as described in the Examples. Each image (462 m×346 m size) was divided into sub-images of size (50 m×50 m) and the mean nuclear size was computed. This step ensured that the entire image was sampled with uniform sampling interval. Thus every data point in the FIGS. 3a & 3b represents mean nuclear size in the predefined sub-image regions. Statistical data from six representative pair of normal and tumor regions are presented in (a). As can be seen, the observed difference in mean nuclear size in the normal and tumor regions was found to be statistically significant. However, the estimated sensitivity and specificity values from these data were only 85% and 62% respectively (c). In order to remedy this problem, the nuclear area fraction (Af) was measured, which parameterizes a combination of nuclear size and number in a given region-of-interest. Statistical comparison of measured area fractions is shown in (b) and the corresponding sensitivity/specificity comparison is shown in (d). These results suggest that it is possible to achieve high sensitivity and high specificity in tumor diagnosis based on nuclear area fractions. The nuclear size criterion can be made highly specific at the cost of decreasing sensitivity.

FIG. 4: Three-dimensional Nuclear Imaging in Excised Breast Tissues Ex Vivo: In contrast to the thin tissue sections, actual surgical specimens are three-dimensional, turbid tissues. In connection with an embodiment of the invention, (a) Schematic for obtaining 3D (x,y,z) image stacks from excised breast tissues is shown. Image stacks were obtained from each field of view (465×425 μm) for user-defined z-depths (100 μm). This process is repeated at every field of view by translating the imaging stage systematically along the X and Y axes. (b) Representative montages of normal and tumor breast tissues (presented as a z-projection image from 20 images in each field of view; Scale bars=1000 μm). (c) and (d) give the statistically-significant differences in nuclear area fraction and fractal dimension between normal and tumor regions. This statistical significance was computed from the analysis of multiple images from different animals (n=4 rats). As can be seen, both nuclear morphometry (area fraction) and tissue topology (fractal dimension) reliably discriminate the tumor regions from the normal tissue components obtained from the same animal. The apparently higher values of area fraction in normal tissue arise possibly from the other tissue components (ducts and fibrofatty components) in the normal breast of the animal that were stained with DAPI. These regions (marked in red circles) typically contribute to false negative values and can be reliably addressed by increasing the threshold (or cut-off) of the area fraction/fractal dimension parameters in the data acquisition/analysis system.

FIG. 5: Nuclear Morphometry Imaging in Human Tissue Microarray: (a) In connection with an embodiment of the invention, representative images showing the nuclear distribution in normal, human breast (fibrofatty) tissue as well as in three breast carcinoma specimens with varying degrees of aggressiveness are shown. The details of the specimens are given in the accompanying table. (b) Nuclear count and hence the nuclear area fraction increases progressively in accordance with the aggressiveness.

FIG. 6: Mechanism for Data Acquisition in Surgical Tissues: In connection with an embodiment of the invention, (A)-(C) show the various stages of tissue assembly in a scaffold for imaging. Stainless frame may be kept on ice during the entire image acquisition duration in order to minimize tissue damage during the data collection process. (D) shows the macroscope stage side-view with a working distance of 85.5 mm, and (E) shows a schematic of excised surgical tissue with nuclear markers for normal and tumor regions.

FIG. 7: Schematic of the multispectral imaging system involving a strategic assembly of the stereo microscope (Olympus SZX12), a multi-wavelength excitation light source with a monochromator (Polychrome, TTL), the emission acousto-optic tunable filter (Chromodynamics Inc, FL, USA) and a CCD camera (Orca ER, Hamamatsu photonics, USA). Data acquisition and analysis were performed using CDI Invivo software (Media Cybernetics, MD, USA). (b) Representative photographs of the anesthetized rats ˜10 days after the tumor generation. Tumor xenografts were generated in the right breast of the animal so that the left breast served as a non-tumor control in each animal studied. Ex vivo images were obtained by excising the shaved skin and exposing the primary tumor or the metastatic lymph node as shown in the top panel. The white arrow indicates the location of the lymph node around which the in vivo images were obtained. (c) Representative histopathology slides (H&E staining) of the tissue slices obtained from the primary breast tumor tissue and the metastatic lymph node tissue. Scale bars=50 μm. The black arrows indicate the margin between the tumor and normal tissue regions.

FIG. 8:(a)-(d) Representative In vivo spectral reflectance images of the primary breast tumor 10 days after injection. On day 10, the rats were anesthetized and 10% fluorescein and/or 1% lymphazurin was injected right under the breast nipple. After 15 minutes of dye equilibration, tumors were excited with light from the Polychrome light source (450 nm-694 nm in consecutive bandwidths of 20 nm) and multispectral AOTF images were obtained for each excitation band (460 nm-750 nm range; Δλ=20 nm). For every excitation, the first image in the emission window constituted the reflectance image while the rest of the images in the series constituted the fluorescence images. Reflectance images at longer wavelengths (>560 nm) clearly show tumor vasculature details below the shaved skin of the animal. (e) Graphical display of reflectance spectra in normal (left) and tumor (right) breasts from an animal after 10-days of tumor growth. The reflectance spectral signatures for 480, 520 and 580 nm excitation are significantly different between the tumor and normal breasts thereby indicating possibilities for quantitative imaging of tumor-specific signatures in tumor xenografts without surgical incision. The figure also shows the spectral reflectance profiles for blood from the same animal. (f) fluorescence image of tumor vasculature after fluorescein injection in the tumor breast (Scale bar=1 cm) and (g) autofluorescence spectra from tumor and non-tumor breasts (h) representative immunofluorescence image obtained from a section of the breast tumor tissue showing upregulation of the glucose transporters (GLUT-1) that is a measure of tumor aggressiveness in vivo as well as (i) the metastastic potency of these tumor cells (indicated by white arrows) as shown by Akt-Alexa488 immunofluorescence labeling of the blood vessel where tumor cells are found amidst anucleated red blood cells. Scale bars=20 m.

FIG. 9:(a)-(c) Representative spectral reflectance images of the lymph node along with the surrounding fatty tissues isolated from rat model. Scale bars=1 mm. As can be seen from the images and from the accompanying graph (d), the reflectance signal has maxima at 500 nm and 620 nm. The plot shown is an average of reflectance profiles from 12 animals (Mean±SEM). The figure (d) also shows that there is no significant difference in the observed spectral reflectance profile even when live cells (1×10⁶ cancer cells) were injected into the lymph node tissues ex vivo. (e) Average In vivo fluorescence profiles (n=14 rats) obtained after injecting 10% fluorescein dye under the nipple. The comparison of fluorescein profile between normal and tumor-associated lymphatics showed no observable difference. However, when 1% lymphazurin dye was injected under the nipple instead of fluorescein, there was an observable difference in spectral reflectance profiles in the normal lymph nodes in vivo (f).

### DETAILED DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

“Cancer” and “cancerous” refer to or describe the physiological condition in mammals that is typically characterized by unregulated cell growth. Examples of cancer include, but are not limited to, breast cancer, colon cancer, lung cancer, hepatocellular cancer, gastric cancer, pancreatic cancer, cervical cancer, ovarian cancer, liver cancer, bladder cancer, cancer of the urinary tract, thyroid cancer, renal cancer, carcinoma, melanoma, head and neck cancer, brain cancer, and prostate cancer, including but not limited to androgen-dependent prostate cancer and androgen-independent prostate cancer.

“Mammal” as used herein refers to any member of the class Mammalia, including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be included within the scope of this term.

“Tumor,” as used herein refers to all neoplastic cell growth and proliferation, whether malignant or benign, and all pre-cancerous and cancerous cells and tissues.

Diagnostic Methods of the Invention

In the underlying experimental work, by systematic comparison of normal and breast tumor tissues from preclinical animal models of breast carcinoma, the inventor demonstrated that nuclear morphometric parameters (e.g., size and nuclear area fraction) and tissue topology parameter (e.g., fractal dimension) may be used as reliable imaging tools for discriminating normal and breast tissues in vivo, in vitro and ex vivo. In order to confirm the utility of this approach in human specimens (for example in human breast specimens), the inventor carried out similar morphometric analysis in a human tissue microarray with four different cases of breast tumor status. The results indicated that the nuclear morphometry has a systematic dependence on the tumor stage and/or aggressiveness. By extending the scope of the current observations to excised human tissues, rapid assessment of tumor margins may be achieved in intraoperative clinical settings, thereby alleviating the aforementioned problems in clinical management of breast cancer and other forms of cancer.

Accordingly, the invention is directed to methods for detecting tumor margins in subjects in need thereof. The method comprises obtaining/providing a tissue sample from the subject and measuring nuclear morphometric and/or tissue topology parameters. The nuclear morphometric and/or tissue topology parameters from the area/areas of in the tissue sample are compared to the areas surrounding the area of interest in the tissue sample. A difference in the nuclear morphometric and/or tissue topology parameters between the area of interest and the surrounding tissue is indicative of a tumor margin. The claimed methods discriminate between normal tissue and tumor tissue. In an embodiment, tissue specimens (for example lumpectomy specimens) are excised and analyzed to obtain nuclear morphometric and/or tissue topology parameters. In one embodiment of the invention, nuclear morphometric and/or tissue topology parameters are measured using the fluorescence intensity imaging system, using microscopes including but not limited to Nikon AZ100 and Nikon TE2000 and cameras including but not limited to the Nikon Qi CCD camera and CoolSNAP CCD camera. For example, images of the tissue samples from subject are obtained using the aforementioned imaging system. The images are then analyzed to acquire nuclear morphometric and/or tissue topology parameters from areas of interest as well as surrounding area. Nuclear morphometric and/or tissue topology parameters are compared and differences in these parameters between the areas of interest and surrounding areas is used to identify tumor margins. In an embodiment, one skilled in the art may employ any generic fluorescence imaging system that has the following minimal components: an epifluorescence microscope, an excitation light source and a detection camera along with the software for data acquisition and analysis. In another embodiment of this system, one can also use a fiber-optic version. This may involve a fiber optic bundle (both excitation and emission light paths) that has a unique advantage of being flexible for obtaining spectral information from the tissues without the need for an epifluorescence microscope. In a further embodiment, a combination modality of the original epifluorescence microscope description along with the flexible fiberoptic version may tremendously increase the utility of the aforementioned imaging system.

In one embodiment of the invention, the nuclear morphometric parameters are nuclear size, nuclear circularity, nuclear count and/or nuclear area fraction. Nuclear area fraction is a sum of the nuclear area and the nuclear count. In an embodiment of the invention, a higher nuclear count in the tissue sample relative to the surrounding area is indicative of a tumor margin and/or presence of a tumor. In another embodiment, nuclear count increases proportional to the aggressiveness of the tumor. In yet another embodiment, a larger nuclear size in the tissue sample relative to the surrounding tissue is indicative of a tumor margin and/or presence of a tumor. In a preferred embodiment of the invention, higher nuclear area fraction in the tissue sample relative to the surrounding tissue is indicative of a tumor margin and/or presence of a tumor. In an embodiment of the invention, nuclear area fraction may be used as a tumor diagnostic marker. In an additional embodiment, nuclear morphometric and/or tissue topology parameters may be obtained in vitro, in vivo and/or ex vivo.

In a further embodiment of the invention, the tissue topology parameter namely “fractal dimension” (a measure of complexity) can add value to the purpose of tumor margin detection. The accuracy of detecting the nuclear morphometric parameters may be very high when images are obtained using a monolayer of cells on glass coverslips. However, the cell density can be quite high in typical surgically excised tissue specimens, which could further interfere in the interpretations of the nuclear morphometric images/parameters. Owing to high values of cell density in the tissues, it may not be always possible to resolve two neighbor nuclei that are located within the theoretical optical resolution limits (˜0.2 μm). To address this critical issue, the inventor developed a novel parameter (fractal dimension) that is solely based on the tissue topology. This parameter is not limited by the optical resolution limits since the intent is not to resolve the individual nuclei but rather analyze the entire tissue segment (within the imaged field of view) as an aggregate. The rationale is that the tumor regions are expected to have a higher tissue complexity (a direct measure of topological arrangement of high density cells) as compared with the normal tissue regions. Thus the aforementioned nuclear morphometric analysis may be complemented with tissue topology parameter (for example fractal dimension) for increasing the tumor detection accuracy.

The invention further provides methods for detecting a tumor in a subject in need thereof. The method comprises obtaining multispectral reflectance images, at various wavelengths, of an area of interest and of the surrounding area in the subject. The reflectance spectra thus obtained of the area of interest is compared with the reflectance spectra of the surrounding area. A difference between the reflectance spectra of the area of interest and the reflectance spectra of the surrounding area is indicative of the presence of a tumor. In an embodiment, tissue specimens (for example lumpectomy specimens) are excised and analyzed to obtain multispectral reflectance images. In one embodiment, lower reflectance signal/reflectance spectra in the area of interest compared to the surrounding area is indicative of tumor presence. In another embodiment, contrasting agents such as lymphazurin and/or fluorescein may be used. In a preferred embodiment, the multispectral fluorescence images are obtained using lymplazurin as the contrasting agent. In an additional embodiment, the first image of the spectral scan constitutes the reflectance images and subsequent images contributed to fluorescence images. In some embodiments of the invention, multispectral reflectance images may be obtained in vitro, in vivo and/or ex vivo.

Multispectral reflectance images may be obtained by using microscopes including but not limited to an Olympus stereo microscope SZX12 or Nikon TE2000 or Nikon AZ 100 microscopes. In one embodiment, the multispectral reflectance images are obtained using the aforementioned microscopes with acousto-optic tunable filters (AOTF) such as those manufactured by Chromodynamics Inc. Multispectral reflectance images may be collected using cameras such as a CCD camera obtained from Orca-ER, Hammatu Photonics, NJ.

In another embodiment, the multispectral reflectance images are obtained using the aforementioned microscopes with fiber optic probes (for example fiber optic probes from Stellarnet Inc., Florida: Fiber optic spectrometers) which may be connected to the spectral detectors as described above. Multispectral reflectance images may be collected using cameras such as a CCD camera obtained from Orca-ER, Hammatu Photonics, NJ. In some embodiments of the invention, multispectral reflectance images using fiber optic probes may be obtained in vivo, in vitro or ex vivo. In other embodiments of the invention, multispectral reflectance images using fiber optic probes may be obtained intraoperatively.

As described above, the methods of the invention may be performed in vivo, in vitro or ex vivo. In a further embodiment, the methods of the invention may be practiced intra-operatively. In an embodiment, the tissue sample that may be used in the claimed methods include but are not limited to lymph node and/or cancerous tissue of a type selected from the group consisting of breast cancer, colon cancer, lung cancer, hepatocellular cancer, gastric cancer, pancreatic cancer, cervical cancer, ovarian cancer, liver cancer, bladder cancer, cancer of the urinary tract, thyroid cancer, renal cancer, carcinoma, melanoma, head and neck cancer, brain cancer and prostate cancer.

The diagnostic methods of the invention may be used on mammalian subjects, including human, monkey, ape, dog, cat, cow, horse, goat, pig, rabbit, mouse and rat.

Imaging Systems of the Invention

A further embodiment of the invention relates to a mechanism for data acquisition in surgical tissues. Among the features of this aspect of the invention that may be desirable are that: (1) a one-to-one correspondence may be maintained between the excised soft, fresh tissue and the surgical site in the patient's body; (2) a surgeon may excise the tissue and complete the suture orientation of the surgical specimen; (3) the tissue may be kept on a sterile, humidified chamber to make identification marks on a number of standard positions, for instance the medial, lateral superior, inferior, deep and anterior positions of the surgical specimen, e.g., with a (glycerol-based) pathology grade colored ink; and (4) surgical specimens may be kept on ice (or a humidified chamber) to minimize tissue damage during image acquisition.

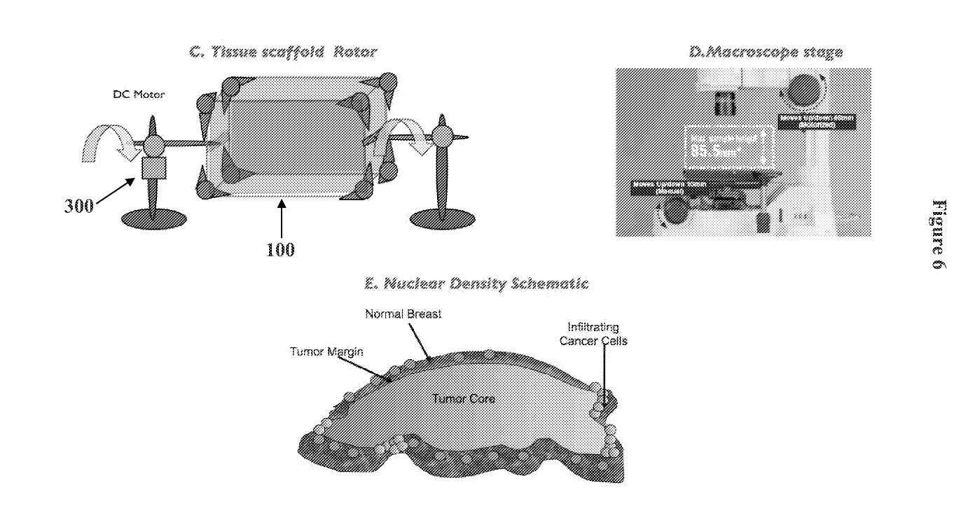

With reference to FIG. 6, an embodiment of the invention provides a surgical tissue scanning scaffold. The invention provides an apparatus to support a tissue sample during data acquisition, comprising a scaffold (100) configured to enclose the tissue sample (200) and a mechanism (300; FIG. 6C) to support the scaffold, adapted to position the tissue sample for optical analysis. In one embodiment, the scaffold is optically transparent. The tissue specimen scaffold may be configured as a cubicle (in one embodiment, with dimensions of approximately 10 cm×5 cm×3 cm, FIG. 6B), and may be composed of a stainless steel frame (400) designed to hold surgical specimens of varying sizes/shapes, rigidly. In an embodiment, the scaffold may be stainless steel with rigid corners and with a slidable cover slip (500). In a further embodiment, the scaffold may be fiber optic with rigid corners and with a slidable cover slip (500).

In some embodiments, the tissue sample size is about 1-5 cc, 5-10 cc, 10-15 cc, 15-20 cc, 20-25 cc, 25-30 cc, 30-35 cc, 35-40 cc, 40-45 cc, 45-50 cc, 50-55 cc, 55-60 cc, 60-65 cc, 65-70 cc, 70-75 cc, 75-80 cc, 80-85 cc, 85-90 cc, 90-95 cc, 95-100 cc. In preferred embodiments of the invention, the tissue sample size is 30-50 cc.

In further embodiments of the invention, the scaffold size is about 1 cm, 2 cm, 3 cm, 4 cm 5 cm, 6 cm, 7 cm, 8 cm, 9 cm or 10 cm larger than the sample tissue. In a preferred embodiment, the scaffold size is about 1 cm larger than the sample tissue.

Those of skill in the art will readily recognize that a variety of configurations, device dimensions and materials may be used in alternate embodiments of the invention. Transparent coverslips (made of, e.g., glass) may help maintain an “optically flat surface” for imaging as well as easy assembly of the surgical tissues in the scaffold. The scaffold may be configured to be attached directly on to the imaging platform stage. In order to minimize the specimen movement and to overcome the rotational errors, the stage may be configured such that it can move along x, y and z directions (only translational) with high precision (˜50 μm) without losing the specimen orientation with respect to the originally initialized scaffold position. Additional elements may be included to facilitate the positioning, rotation, placement or other movement of the scaffold relative to optical analysis equipment.

Every scanned point on the specimen may be assigned an unique set of (x,y,z) coordinates, and these coordinates may be referenced against the initially marked points described above so that every single point on the surgically excised tumor specimen can be mapped with a corresponding point at the surgical site in the patient's body. Scaffolds of a variety of sizes and shapes may be used to accommodate surgical specimens of variable size/shape. Use of the scaffolds may result in improved performance, such as increased scan speed and read-out speed of a photosensor module, and parallel processing of the acquired data and improvising the optical configuration so as to simultaneously collect both spectral and FLIM data from the specimen's fluorescence emission.

In an embodiment of the invention, a protocol for image acquisition allows for positive margins to be identified in surgically excised intact tissue intra-operatively, even before such a specimen may be sectioned by a pathologist. First, the surface of the surgical specimen may be painted with a fluorescence marker specific for nuclear staining (DAPI, Hoechst: Invitrogen) and a fast, nuclear grade imaging data set may be carried out from the entire specimen. For every field of view, this data set will comprise of z-stacks of (x,y) images with a user-defined choice of scan speed, spatial resolution and thickness of the tissue (i.e., z-stack depth) that has to be imaged. At the end of the first-step scan, the computer software will carry out a rapid, image-segmentation process to identify regions where the nuclear grade (number and the size of the nuclei in a user-defined volume) is significantly higher. These regions may be marked as “Suspected Lesion Clusters.” Since the scanning system assigns unique (x,y,z) coordinates to every single point on the surgical specimen, these lesion clusters are assigned unique volume labels in the computer memory. Thus, through use of the system and method of the invention, the tumor margins can be identified intra-operatively.

Advantages of the Invention

The invention relates to a method and system for tumor margin detection based on nuclear morphometry and tissue topology. For example nuclear morphometry parameter such as nuclear area fraction provides consistent and significant difference between normal and tumor tissue, and it also yields high sensitivity and specificity in the analysis of specimens with both normal and tumor regions. Therefore nuclear area fraction is an important diagnostic parameter.

Further, the invention may enable surgeons to identify tumor margins in surgically resected specimens intraoperatively. By fast imaging of the surgical specimens labeled with, for example, nuclear dyes, the invention may provide for rapid assessment of tumor margins while a patient is in the operating room so that surgeons can make informed decisions as to the further steps in a surgical or other procedure; for example, whether additional tissue should be removed from the patient's body to ensure that the tumor is removed completely. Moreover, in various embodiments, the inventive methods and systems may be applicable for identification of tumor margins regardless of the type of tumor. Various types of tumors and cancerous tissues that may be examined in accordance with alternate embodiments of the invention will be readily apparent to those of skill in the art and can be used in accordance with the present invention by mere routine experimentation. Any tumor or cancerous tissue that is surgically resected or otherwise obtained may be used in connection with alternate embodiments of the invention.

Moreover, among the advantages of the present invention is that, in various embodiments, the invention may reach single cell resolution so that within the time constraints in the operating room, it is possible to identify even small clusters of cancer cells. Current approaches suffer from poor sampling, wherein only a small section of a resected specimen is analyzed. On the other hand, the inventive imaging approach may scan the entire specimen so that all specimens may be sampled and parameters such as nuclear area fraction, assessed.

### EXAMPLES

The following examples are provided to better illustrate the invention and is not to be interpreted as limiting the scope of the invention. To the extent that specific materials are mentioned, it is merely for purposes of illustration and is not intended to limit the invention. One skilled in the art may develop equivalent means, devices or reactants without the exercise of inventive capacity and without departing from the scope of the invention.

### Example 1

Experimental Methods of the Invention

Cell Culture & Tumor Generation in Rats

Adult female Fisher 344 rats (180-210 g body weight) were used in the current studies. MAT B-III rat breast cancer cell line was purchased from ATCC (Manassas, Va., USA) and cultured in McCoy's 5a medium supplemented with 10% fetal bovine serum. When confluent, cells were harvested and washed twice with PBS, counted with trypan blue staining for viability. In order to generate breast tumor xenografts, the rats were anesthetized by maintaining a steady stream of oxygen/isoflurane using a nose cone/face mask. After removing the hair and sterilizing the skin, 10⁶ cell/0.2 ml were injected subcutaneously into the mammary fat pads under the rat's nipple on the right breast. Left breasts without tumor cell injection served as normal control for every animal. All experiments were conducted on both left (normal) and right (tumor) breasts in each animal. Rats were observed at set intervals (days 0,1,3,5,7,9,11,13,15 and 21) for tumor growth. It was observed that the above inoculation protocol generated tumors (100% efficiency) within 2 days and the tumor size reached typically 2-4 cm in 3 weeks. All procedures used were carefully controlled to adhere to the approved animal protocols (Cedars-Sinai Medical Center, Institutional Animal Care and Use Committee).

Adult female Fisher 344 rats (-180-210 g body weight) were used in these experiments. MAT B-III rat breast cancer cell line was purchased from ATCC and cultured in McCoy's 5a medium supplemented with 10% FCS. In order to generate breast tumor xenografts, the rats were anesthetized by maintaining a steady stream of oxygen/isoflurane by setting up a nose cone/face mask. After removing the hair and sterilizing the skin, 10⁶ cell/0.2 ml were injected subcutaneously into the mammary fat pads under the rat's nipple. Rats were observed periodically for tumor growth. We observed that the above inoculation protocol generated tumors (100% efficiency) within 2 days and the tumor size reached typically 2-4 cm in 3 weeks. All procedures used were carefully controlled to adhere to the approved institutional animal (IACUC) protocols.

Image Acquisition

A wide-field fluorescence microscopy imaging system (Nikon TE2000; CoolSNAP CCD camera) was employed in collecting all the images. This system utilizes the mercury arc lamp for excitation and appropriate filter cubes for collecting fluorescence from the specimen (DAPI filter: 360/40 nm excitation; 400 nm LP dichroic; 460/50 nm emission & Alexa 488 filter: 480/30 nm excitation; 505 nm LP dichroic; 535/40 nm emission). An automated stage-scanning feature of the imaging system enabled the rapid acquisition of data along both X and Y axes. After three weeks of tumor growth, animals were anesthetized and tumor tissues were excised and immediately stored in formalin containers. In order to obtain a matched pair of breast specimens without the tumor, mammary fat pads and the surrounding breast stroma were also collected from the left breast (no tumor injection) of each animal. For this study, twelve animals were subdivided into two groups: group 1 (n=6) animal tissues were used in making paraffin blocks and subsequent thin tissue sectioning (5-10 microns thickness), and group 2 (n=6) animal tissues were used as thick tissue specimens (˜4 cm volume) for three-dimensional imaging as described in the next section. The goal was to demonstrate the inventive method of nuclear morphometry analysis in thin tissue sections (group 1) as well as in realistic thick breast tissues that mimic the surgical specimens (group 2). Since the purpose of this study was to evaluate the rapid assessment of nuclear architecture in tissues, the inventor chose to use a DNA intercalating fluorescent dye, DAPI (Invitrogen, Carsbad, Calif., USA), that has bright fluorescence for fast imaging of nuclear-specific fluorescence from the breast tissues. The DAPI labeling protocol was optimized for good signal-to-noise ratio as well as for rapid readout of the images. It was found that both the thin tissue slides and the thick tissue specimens could be labeled rapidly (˜3 minutes, room temperature, 50 ng/ml working concentration) for optimal imaging. Supporting immunofluorescence studies were carried out by labeling the group 1 tissue sections with cancer-specific primary antibodies (rabbit polyclonal) raised against key metabolic targets Glucose transporter 1 (GLUT1), epidermal growth factor receptor (EGFR), fatty acid synthase (FAS) and Akt (Abeam, Cambridge, Mass., USA). Fluorescence visualization of the tissue slides was enhanced by secondary antibodies conjugated with Alexa 488 fluorophore. Human tissue microarrays (US Biomax Inc, MD, USA) were labeled with DAPI and cell proliferation marker, Ki67 tagged with Alexa 488 fluorophore. Data acquisition was facilitated by the QED Invivo Software (Media Cybernetics Inc., Silverspring, Md., USA). Serial images along X,Y were obtained and tiled together to obtain the complete image of the entire specimen. Three-dimensional stacks of images were obtained by collecting series of XY images over a defined Z-depth range (˜100-150 microns). Typical time of acquisition per image (1392×1040 pixels) was under 2 seconds.

Macroscopic Spectral Imaging in Vivo

An Olympus stereo microscope was used for obtaining macroscopic spectral images ex vivo and in vivo. For exciting the rat breast tissue in live animals, a single-mode optical fiber was attached to a high-power arc lamp source with an in-built monochromator (Polychrome V, TTL photonics). This source offers variable excitation wavelengths (280 nm to 694 nm) so that the entire spectrum of fluorophores in the visible (and UV) range can be easily excited.

On the detection side, we attached a acousto-optic tunable filter (AOTF, Chromodynamics) for collecting reflectance/fluorescence emission from the tissues at specified bandwidths over a broad wavelength range (460 nm-1200 nm) [43, 44]. A traditional spectrometer (such as the Stellarnet fiberoptic spectrometer described above) collects spectral data over a defined wavelength region (typically 280 nm-900 nm). This is a simple implementation of obtaining spectral data from a single point (pixel). Alternately, if one wants to obtain such wavelength-resolved information from all the pixels in a 2D image, then one may use a “spectral imaging camera”. AOTF is one such device which facilitates this spectral imaging facility. Another commercially available spectral imaging camera is from the CRi (Cambridge Research Systems: Nuance Camera). Spectrally-resolved full-field images were collected by a CCD camera (Orca-ER, Hamamatu Photonics, NJ). Data acquisition and analysis were facilitated by CDI software (QED imaging, Media Cybernetics). Precautions were taken to maintain the body temperature of the rats during the experiments by placing the animal on a heating pad. Rat's limbs were fixed by the adhesive tapes and the body position was very carefully kept under the imaging detector. Respiratory rates were closely monitored by adjusting the concentration of inhaled oxygen/coinsurance mixture, usually at the rate of 30-60/minute. For every excitation wavelength, a complete emission spectral scan was carried out (460 nm-750 nm; 20 nm steps). Typically the first image of the spectral scan constituted the reflectance image and the subsequent images contributed to the fluorescence images. After collecting these spectral images from both the breast and axilla, 50 μl of fluorescein (10% w/v in PBS) or 1% lymphazurin was injected subcutaneously into the mammary fat pads or tumors under the nipple using the insulin syringe. The above imaging experimental session was carried out at various time points (5, 7, 10, 14, 21 days after cell injection) during the tumor growth. Beyond 21 days, the tumor size became too big and there were signs of ulceration. Therefore we euthanized the rats according to the standard procedures outlined in the institutional IACUC protocol. The tumors, lymph nodes on both sides of the rat were dissected and stored in formalin.

H & E Pathology Analysis

Standard pathology slides were prepared from representative breast tissues and axillary lymph node tissues and fixed in formalin immediately after harvesting from the rats. Later these tissues were paraffin fixed and sectioned (5-10 microns) in a microtome for routine H&E staining and visualization.

Immunofluorescence Studies

Deparaffinized breast tissues were labeled with primary antibodies (rabbit polyclonal) raised againt key metabolic targets such as Glucose transporter 1 (GLUT1), epidermal growth factor receptor (EGFR), fatty acid synthase (FAS) and Akt. These molecules have been known to be critical in regulating glucose metabolism in breast tumor tissues. Fluorescence visualization of the tissue slides were enhanced by secondary antibodies conjugated with Alexa 488 fluorophore. A widefield fluorescence imaging system was employed in imaging these slides.

Data Analysis

Tissue fluorescence images obtained by the aforementioned protocols were analyzed for three morphometric parameters; namely, nuclear size, circularity and nuclear count. The rationale behind choosing these parameters is the fact that tumors are most commonly associated with increased cell proliferation as compared with the non-neoplastic (normal) regions which in turn, leads to a higher nuclear density as well. The inventor sought to evaluate the feasibility of quantitative characterization of nuclear architecture (as exemplified by the three parameters described above) in breast tumors. To this end, the inventor applied a well-known algorithm namely, the Watershed Algorithm—for automatic estimation of nuclear size and count in the fluorescence images obtained. The Watershed algorithm is one of the many methods of image segmentation (i.e., the process of partitioning a digital image into multiple segments (sets of pixels)) [21-23]. The watershed transformation considers the gradient magnitude of an image as a topographic surface. Pixels having the highest gradient magnitude intensities correspond to watershed lines, which represent the region boundaries. Water placed on any pixel enclosed by a common watershed line flows downhill to a common local intensity minimum. Pixels draining to a common minimum form a catch basin, which represents a segment. In the present invention, this approach was expected to segment the nuclear fluorescence images and extract the statistics such as nuclear size and count. The inventor used a custom-plugin written in the ImageJ (NIH) program for the watershed analysis of the images (available at http://rsbweb.nih.gov/ij/). The inventor further tested another equivalent approach for achieving automated nuclear statistics based on the topology of the digital images by the CellAnalyst software program (available at http://www.assaysoft.com) [24-28]. In this approach, an image pixel is defined to have 4 vertices (corners), 4 edges, and one face. Algebraic topology uses algebraic operations with these objects to capture and count the number of completed cycles—circular sequences of edges. The completion of a cycle indicates the presence of a cell (or nuclei in this case). The topological nature of the algorithm makes it especially suitable for nuclear counting since (a) the count of nuclei is independent of their locations, (b) the measurements of nuclei are independent of their orientations with respect to the image grid, and (c) the nuclei and other features are captured with no deformation, smoothing, blurring or approximation. In order to evaluate if the difference in nuclear morphometry is significant enough to serve as a reliable diagnosis criterion in situations that mimic the intraoperative settings, the inventor also computed the Nuclear Area Fraction in each image by using a particle analyzer plugin written in the ImageJ software (available at http://rsbweb.nih.gov/ij/). This parameter yields a comprehensive picture of nuclear distribution that takes into account both the nuclear size/shape and the nuclear count. Finally, in order to measure the complexity in the tissue images, the inventor also measured an important topological parameter—“fractal dimension”—which measures the degree of connectedness. Fractal is typically a rough and geometric shape that looks almost identical at arbitrarily various levels of magnification. This feature stems for the principle of self-similarity and is a defining characteristic of the spatial complexity. For the present purpose of understanding complex, highly-connected nuclear architecture in the fluorescence images of the breast tissues, it is possible to quantify the tissue complexity by measuring the fractal dimension [29-32]. Fractal dimension, D, is a statistical quantity that gives an indication of how completely a fractal appears to fill space, as one zooms down to finer and finer scales. The inventor chose to measure the fractal dimension to investigate if this parameter can be a robust indicator of the breast tumor tissue complexity and if this parameter can also serve as a reliable diagnostic criterion for margin assessment. This was measured by box-counting algorithm written and available in the ImageJ software.

Statistical Analysis

Morphological and topological data set from normal and tumor specimens from both Group 1 and Group 2 were analyzed for statistical significance by performing Students' t-test (unpaired set with equal variance). In each group, specimens from at least five different animals were included to address the issue of variations from animalto-animal. The data presented herein had a p value, p<0.0001.

### Example 2

Nuclear Morphometric Parameters Discriminate Normal and Tumor Tissues in Vitro

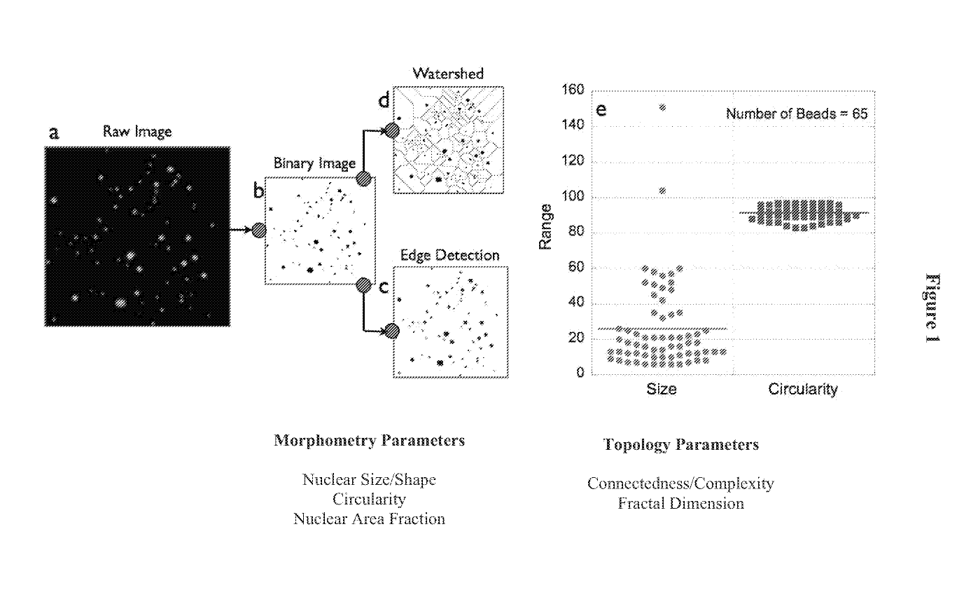

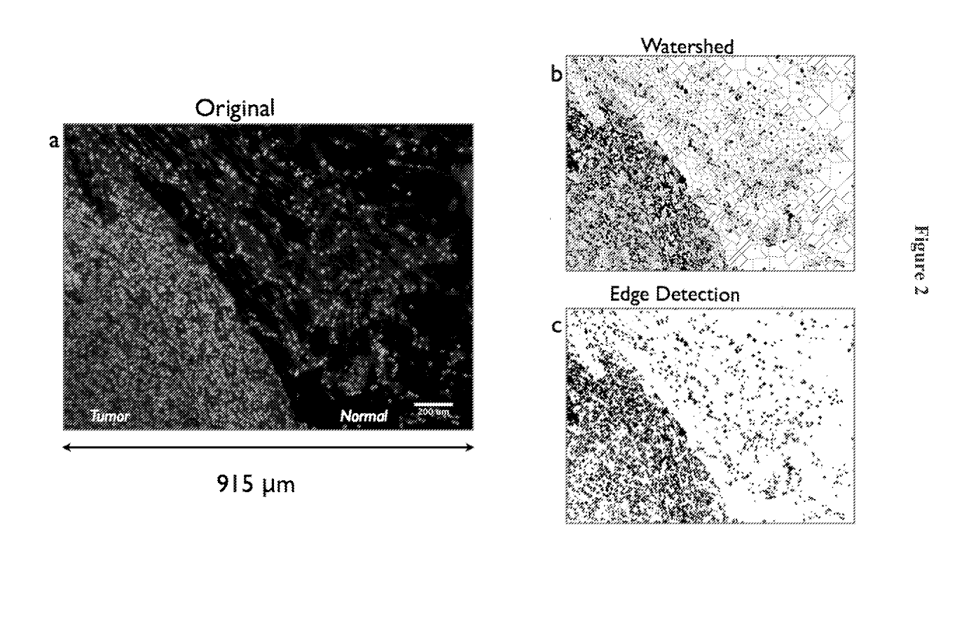

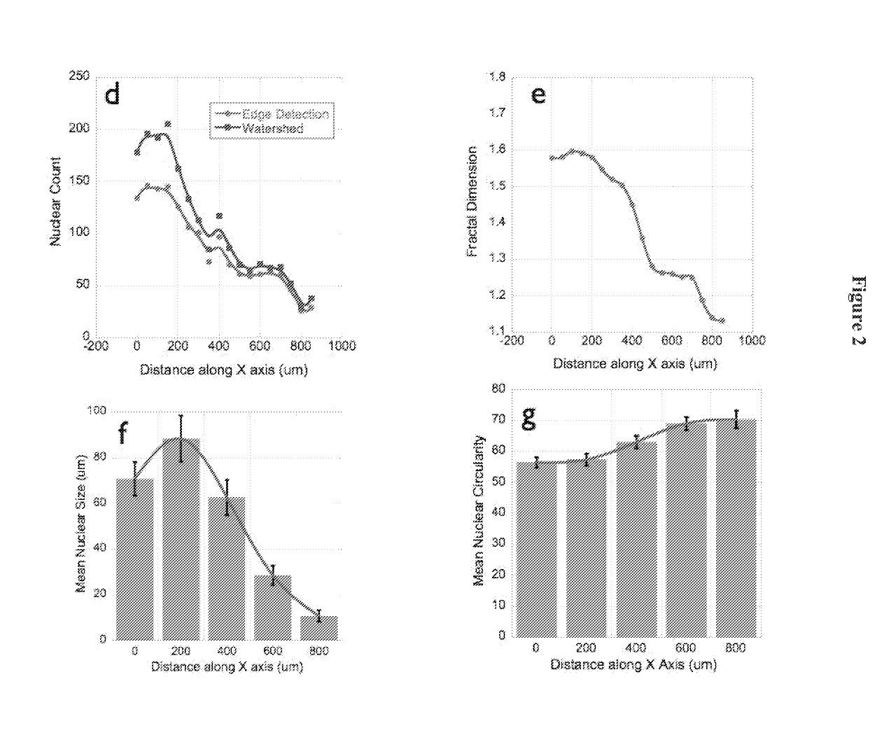

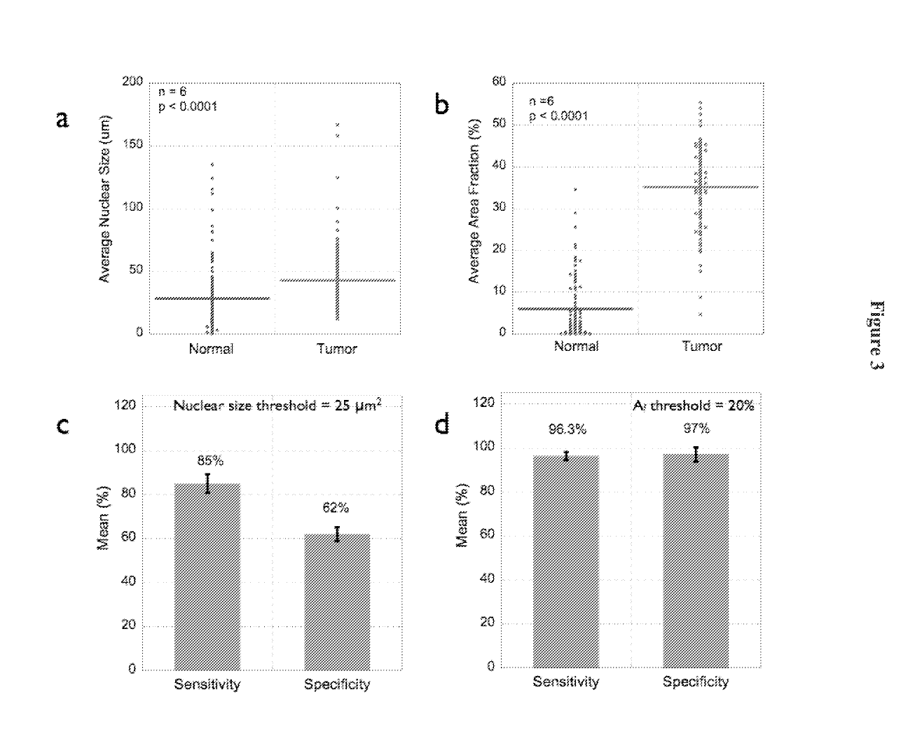

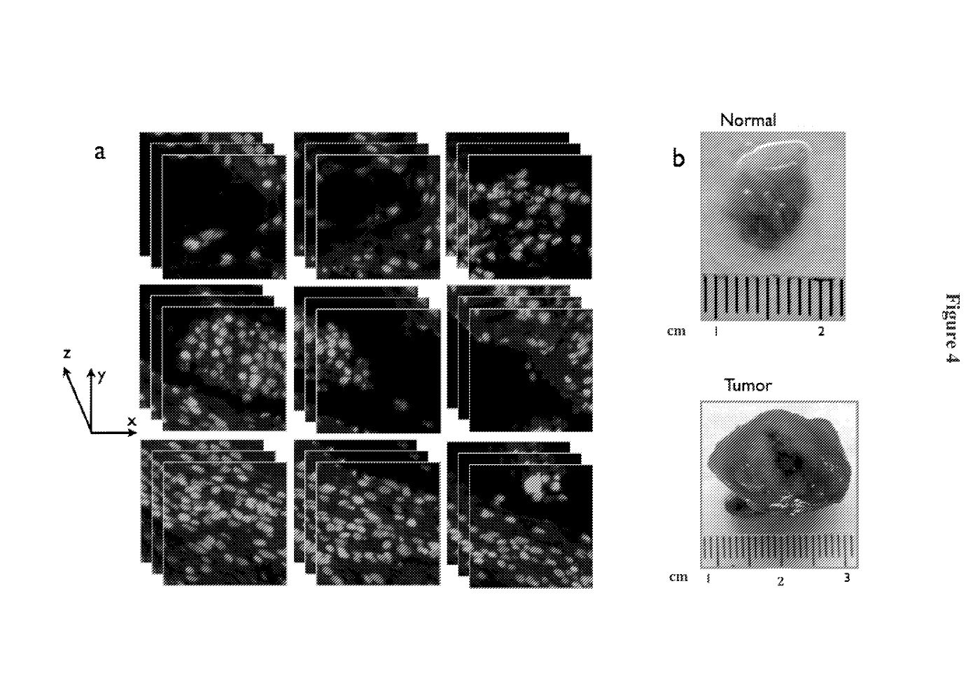

The basic premise of nuclear morphometry analysis is demonstrated in FIG. 1, which shows certain steps involved in extracting information (nuclear size/shape, count, etc.) from the raw fluorescence image. A breast tissue is inherently heterogenous since it is composed of multiple cell types (e.g., epithelial, fibroblasts, endothelial and fatty tissue components) and the resulting nuclear architecture can be fairly complex. It was therefore considered important to validate the proposed nuclear morphometry analysis to confirm the variability in analysis and the statistical significance of the extracted parameters. FIG. 1a shows a representative two-dimensional image of fluorescent microbeads of different sizes and shapes. Image processing (binary threshold) and image segmentation steps as demonstrated in FIG. 1b-1d yield the required nuclear parameters. The inventor next tested whether the proposed nuclear morphometric parameters can reliably discriminate tumor margins in breast tissue specimens. In order to do this, the inventor first chose thin sections of tissue specimens that were known to contain tumor regions bordering with normal epithelium. Watershed and Edge detection analysis were carried out on this set of specimens as follows: individual images of 915×686 μm size were subdivided into regular image units of 50×686 μm size. Nuclear morphometric parameters were calculated on these individual image units. A representative data set and the associated analyses are presented in FIG. 2: nuclear size and count systematically decrease as one moves from tumor-rich regions to normal-only regions, as graphically illustrated. Normal breast regions tend to have smaller nuclear size and lesser nuclear count as compared to the tumor-filled breast regions. In contrast to the above two parameters, nuclear circularity does not exhibit a significant difference between normal and tumor regions. In light of this observation, the inventor chose not to include nuclear circularity in the later analysis of breast tissue morphometry. The increase in nuclear density in tumor-rich regions of the tissue poses another technical challenge in the analysis of nuclear morphometry. In some regions, as can be seen in FIG. 2a, the overlap of the neighboring nuclei is high enough to introduce artifacts in nuclear counting since this may exceed the best optical resolution that can be achieved (˜0.20 μm). This potentially underestimates the resulting nuclear count. Although this is an inherent limitation of optical imaging methods, one can also derive another useful topological parameter from this situation: in tissue images with high degree of overlap between individual nuclei (or cells in general), a topological survey can be performed by measuring the degree of connectedness or nonlinearity in the images. By measuring the fractal dimension of these images (as described in various Examples above), one can infer the extent of complexity in the images. The inventor computed the fractal dimension in the individual image subunits as described above. FIG. 2e demonstrates that the computed fractal dimension changes from 1.6 (tumor) to 1.2 (normal) mimicking the spatial profile of the nuclear morphometry (size and count) parameters. This feature was observed in all the images analyzed. Having shown that nuclear morphometry and tissue topology analysis can yield a robust measure of the spatial transition from normal to tumor regions in breast tissue sections, the inventor then analyzed multiple sets of images from normal and tumor tissue sections obtained from different animals with varying stages of tumor growth. A rigorous statistical analysis of all the morphometric and topological parameters was carried out. For clarity, a representative statistical analysis of nuclear size is given in FIG. 3a. As can be seen, the mean nuclear size was found to be statistically different between normal and tumor tissue sections.

In a typical lumpectomy procedure, the surgeon is guided by preoperative radiological images of the tumor for locating the tumor in the patient's breast and for removing the tumor and the surrounding normal tissue. The immediate question is how much of this excised tissue is clear of tumor cells in the periphery. It is useful to have a specific diagnosis criterion that could potentially enable the surgeon in answering the above question. Based on our statistical results from FIGS. 2 and 3a, the inventor investigated if the nuclear size could be such a diagnosis criterion. This was tested by analyzing the tissue sections (n=6) that contained both normal and tumor regions in the same field of view, as exemplified in FIG. 2a. By using a diagnosis criterion based on the nuclear size threshold of 25 μm² (as obtained from FIG. 3a), the inventor computed the sensitivity and specificity in detecting tumor regions within a normal breast tissue (Table 1).

TABLE 1
Sensitivity and Specificity calculations based on two diagnosis criteria.

| | | Nuclear Area |
|---|---|---|
| | Nuclear Size | Fraction |
| Diagnosis Criterion | Threshold = 25 μm² | Threshold = 20% |
| True Positive | 138/330 (41.1%) | 49/82 (59.8%) |
| (Tumor identified as Tumor) | | |
| False Positive | 16/330 (4.8%) | 1/82 (1.2%) |
| (Normal identified as Tumor) | | |
| True Negative | 92/330 (27.8%) | 30/82 (36.5%) |
| (Normal identified as Normal | | |
| False Negative | 84/330 (25.5%) | 2/82 (2.4%) |
| (Tumor identified as Normal) | | |
| Sensitivity = | 85.0 ± 2.5% | 96.3 ± 1.5% |
| [True Positives/(True | | |
| Positives + False Negatives)] | | |
| Specificity = | 62.5 ± 2.5% | 97.0 ± 2.0% |
| [True Negatives/(True | | |
| Negatives + False Positives)] | | |

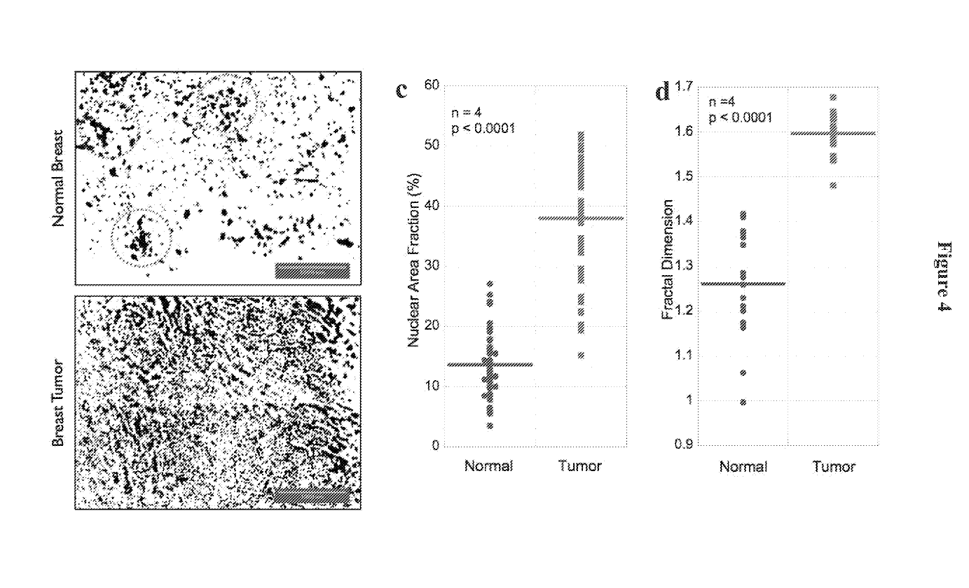

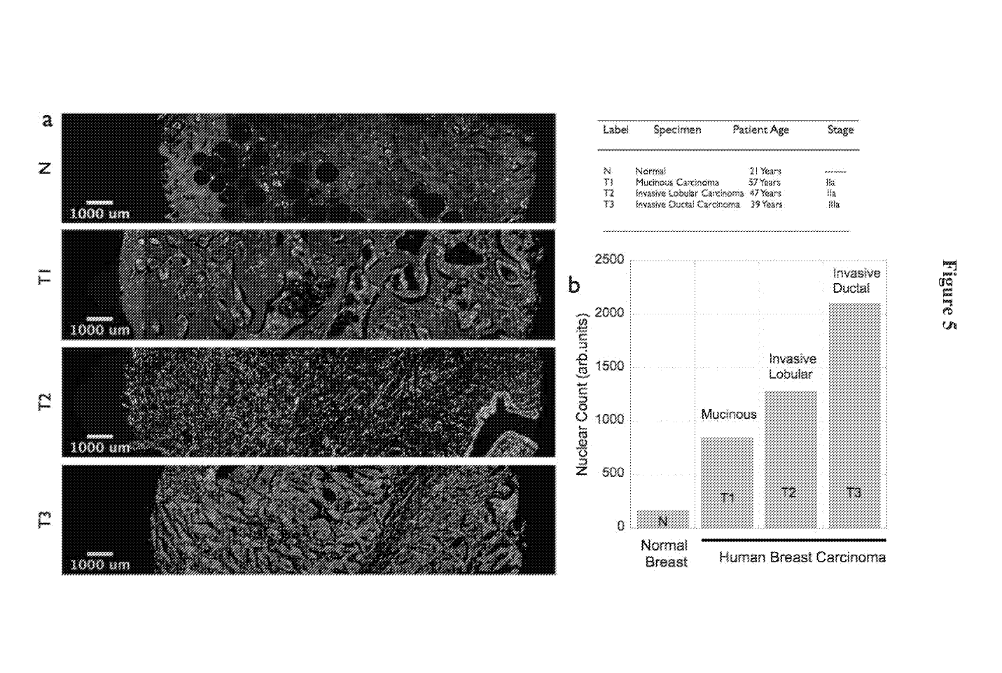

In a binary classification scenario where the goal is to detect tumor regions (true positive) in an otherwise normal tissue periphery (true negative), sensitivity is the statistical measure of the proportion of true positives that are correctly identified and specificity is the corresponding statistical measure of the proportion of the true negatives that are correctly identified. This analysis is summarized in FIG. 3c, where the sensitivity and specificity of detecting tumor regions were 85% and 62.5% respectively. Although the difference in nuclear size was found to be statistically significant, and thus this criterion might be used, it may not be the best diagnosis criterion for implementing in an intra-operative setting. However, during the course of the underlying studies, the inventor found that nuclear area fraction (which is a combination of nuclear size and count) provided not only a statistically significant difference between normal and tumor regions (FIG. 3b) but also yielded a very high sensitivity and specificity in the analysis of specimens with both normal and tumor regions (FIG. 3d). This can be a simple, reliable, and reproducible diagnosis criterion that can be implemented in tumor margin detection in excised tumor tissues. In order to test this in more realistic (thick) breast tissues, the inventor performed morphometric and topology analysis in Group 2 specimens as mentioned above. FIG. 4a shows the schematic of 3D data acquisition. Representative montages of large field of view of normal and tumor breast tissues show that the nuclear count is significantly higher in the tumor tissue as compared with the normal counterpart. Computation of nuclear area fraction and fractal dimension in multiple specimens demonstrate the feasibility of applying this proposed morphometric/topological approach even thick excised tissues.

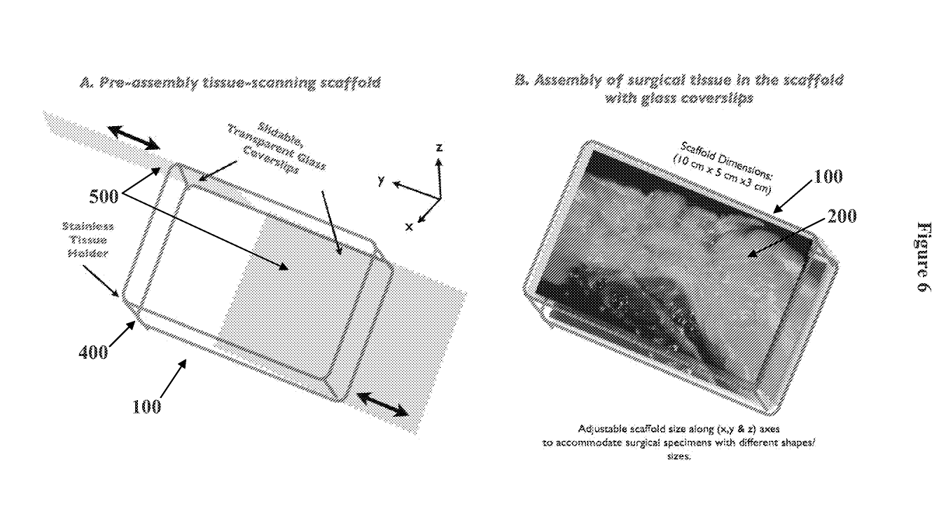

Finally, the inventor extended the scope of preclinical observations to human breast tumor cases where it was examined if the proposed nuclear morphometry analysis would give insight into the various tumor stages and/or aggressiveness. FIG. 5 shows representative nuclear fluorescence images on a tissue microarray (US Biomax Inc., #T085) labeled with cell proliferation marker (Ki67) and nuclear marker (DAPI). As can be seen from FIG. 5b, nuclear count systematically increases in proportion to the aggressiveness of the breast cancer. As it is evident from the images, the nuclear grade (heterogeneity in nuclear size and shape) is also significantly different in breast carcinoma as compared with normal breast tissues thereby offering additional quantitative measures for rapid diagnosis in intra-operative settings.

### Example 3

The inventor demonstrated the utility of measuring nuclear morphometric and tissue topology parameters in discriminating normal and tumor tissues in a rat model of breast carcinoma. The rationale behind this study is based on the drastic increase in cell proliferation that accompanies tumorigenesis. The invention involves a novel and robust image analysis concept that can be employed in a practically platform-independent manner. In earlier studies and even in current practice of tumor histopathology, it is a commonplace observation that nuclear-to-cytoplasmic ratio increases in specimens obtained from breast tumors. However, while translating this observation to tissue specimens with both normal and tumor regions (as judged by immunofluorescence studies, data not shown), the inventor concluded that nuclear size as a diagnostic criterion may not yield good enough sensitivity and specificity in reliably delineating tumor regions in an otherwise normal breast tissue. While not wishing to be bound by any particular theory, the inventor's data suggests the preclusion of nuclear size as a reliable diagnostic criterion for tumor margin assessment. On the other hand, nuclear area fraction addresses this issue very effectively since it is a combination of both nuclear size and count in any given region of the analyzed image, and thus yields high sensitivity and specificity (˜97%) in tumor detection. This is further substantiated by an independent parameter, fractal dimension, based on the tissue topology.

The results also point to the fact that the inventive diagnostic criterion is applicable not only in thin tissue sections but also in realistic thick excised tissues. The CFI Plan Fluor DLL 20× (Nikon; 0.50 numerical aperture; 2.10 mm working distance) objective lens used in the underlying study allowed the inventor to reproducibly obtain fluorescence signals up to 1.60 mm of the thick tissue sections. This reduction in “effective” working distance (as compared to the expected 2.10 mm for the objective lens) can be attributed to tissue absorption, shorter excitation wavelengths (˜350 nm) as well as multiple scattering events in the tissue sections.

Although this may limit deeper penetration, the measurable tissue depth (˜1.60 mm) is more than the typical depth (˜1 mm) where the positive tumor margin is typically defined.

Finally, data on human tissue microarrays further suggest that it is also possible to extend the scope of the proposed diagnostic criterion from tumor margin detection to preliminary tumor staging in operating rooms. The inventive method can rapidly give a spatial map of nuclear distribution in the excised tissue from which one can obtain information on potential “tumor-like” regions on the surface of the surgical specimen. To increase the precision in margin assessment, it is possible to label these “tumor-like” regions with cancer-specific antibodies tagged with fluorophores—without compromising the intraoperative diagnosis features (e.g., speed, sensitivity and specificity) of the nuclear architecture imaging. Appropriate specimen handling strategies may be important to implement the invention in intraoperative settings to avoid commonly encountered problems such as specimen shrinkage and related artifacts [33, 34]. (One such strategy is described in this application).

### Example 4

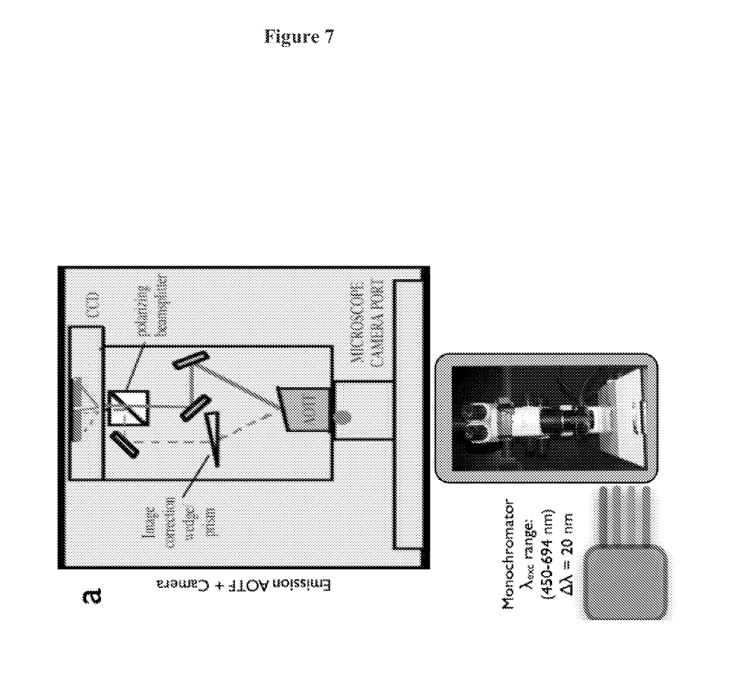

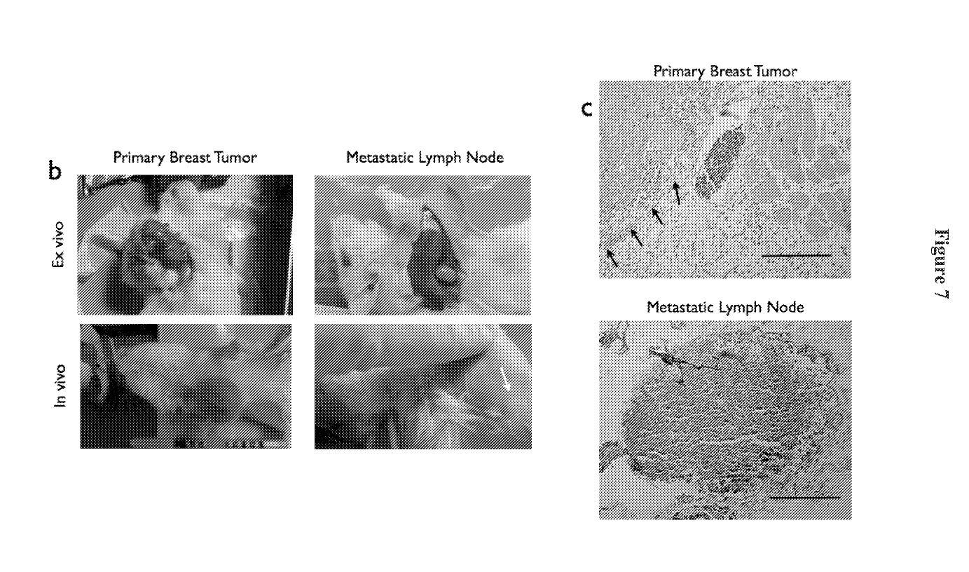

FIG. 7 shows the schematic of the imaging system with the acousto-optic tunable filter. Respiratory rates were closely monitored by adjusting the concentration of inhaled oxygen/coinsurance mixture, usually at the rate of 30-60 per minute. For every excitation wavelength, a complete emission spectral scan was carried out (460 nm-750 nm; 20 nm steps). Typically the first image of the spectral scan constituted the reflectance image and the subsequent images contributed to the fluorescence images. After collecting these spectral images from both the breast and axilla, 50 μl of fluorescein (10% w/v in PBS) or 1% lymphazurin was injected subcutaneously into the mammary fat pads or tumors under the nipple using the insulin syringe. The above imaging experimental session was carried out at various time points (5, 7, 10, 14, 21 days after cell injection) during the tumor growth. Beyond 21 days, the tumors became larger (>4 cm) and there were signs of ulceration. Therefore the rats were euthanized according to the standard procedures outlined in the animal protocol. The tumors, lymph nodes from right and left sides of the rat were dissected and stored in formalin. Supporting measurements were carried out from these fixed tissues by standard histopathology and immunofluorescence imaging. All the analyses presented in this study correspond to primary breast tumors and metastatic lymph nodes as confirmed by histopathology. Representative hematoxylin and eosin stained images are shown in FIG. 7c.

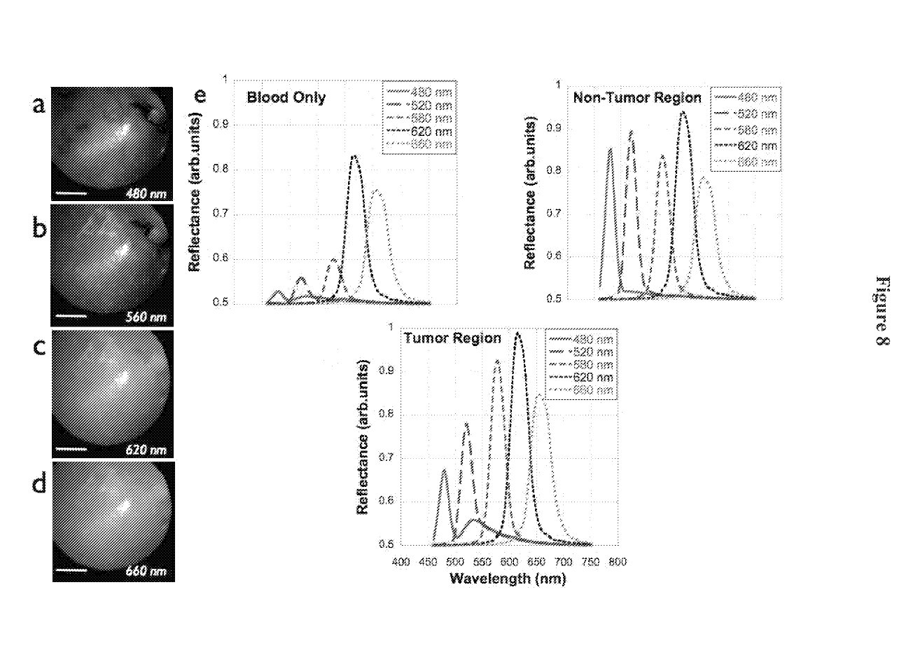

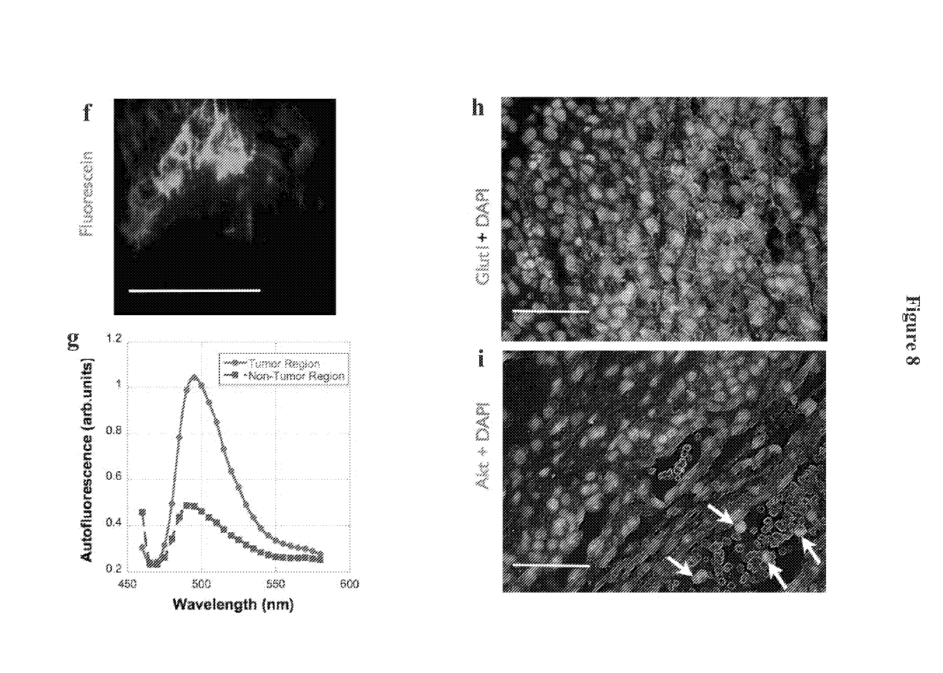

The inventor first tested if the experimental design could distinguish the primary breast tumors from non-tumor regions as well as from the surrounding autofluorescence and/or vasculature. FIG. 8(a-d) shows spectral reflectance images of the rat breast with 3-week old primary tumor. A spectral emission scan from 480 nm to 694 nm yielded high contrast in visualizing the tumor, vasculature at varying depths without any surgical exposure of the tumors. The obtained images confirmed visualization of tumor vasculature from beneath the rat skin for longer wavelengths (>600 nm).

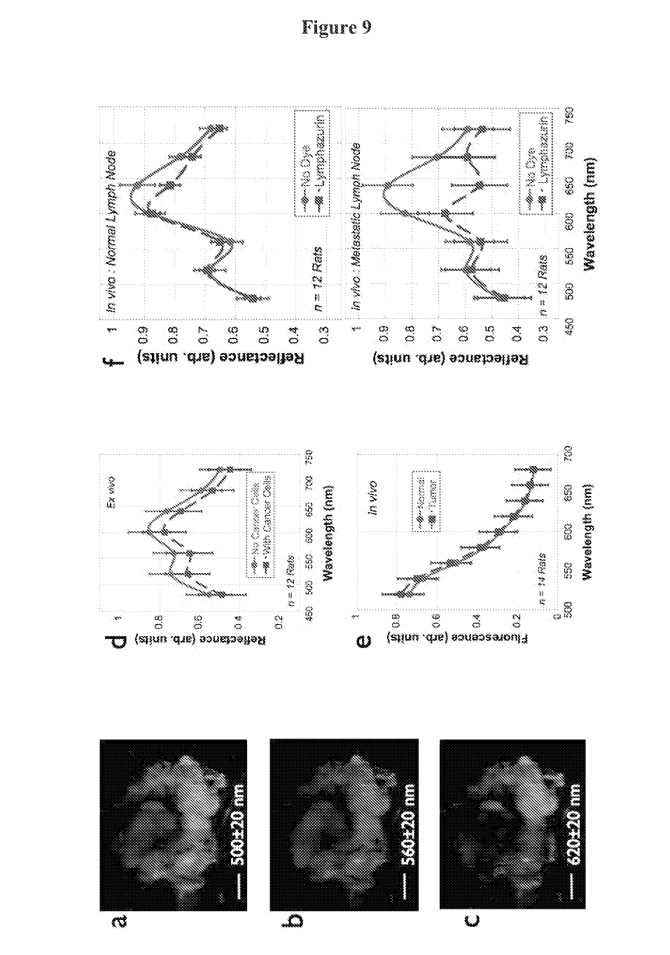

A quantitative analysis of the various spectra is shown in FIG. 8e where the spectral reflectance and fluorescence signatures of the tumor regions were compared with those of the non-tumor regions (left breast of the same animal in each case). The reflectance signals were significantly lower in tumor regions in the spectral region 460 nm-550 nm as compared to the non-tumor regions and these observed differences were reproducibly the same in each animal that was studied. Interestingly the autofluorescence signals measured in the tumor region (excitation 480 nm; emission ˜520 nm) were significantly higher in the tumor regions as can be seen in FIG. 8e as well as FIG. 8g. No observable autofluorescence signals were present in the spectral regions beyond 560 nm. Earlier studies have found that tumor growth also leads to irregular vasculature that is observably different from normal vasculature. In addition to fluorescence visualization of tumor vasculature, it would be valuable to understand the various components of vascular network in vivo. FIG. 8e also shows the spectral reflectance profiles of only blood. Low reflectance signals in the spectral region 450-600 nm for the blood further confirms that the observed difference between tumor and non-tumor regions arose clearly from the physiological changes in molecular composition in the tumor rather than from the modified vascular network. In fact, a careful comparison of the tumor and non-tumor regions in spectral reflectance profiles in the spectral region beyond 600 nm indicate that both these signatures are in good agreement with those of only the blood component. FIGS. 8h and 8i show the aggressiveness (GLUT1 over expression) and the metastatic potential (circulating tumor cells) of the primary tumor. The inventor tested if this metastatic potential could be detected by spectral reflectance/fluorescence imaging in the lymph nodes as well. FIG. 9(a-c) shows the surgically excised fresh axillary nodes with the surrounding fatty tissue ex vivo. Attempts to inject live MatBIII cells into the surgically excised lymph node tissues (akin to ex vivo implantation) did not yield any significant difference in spectral reflectance signatures as can be seen in FIG. 9d. Efficacy of fluorescein in providing better sensitivity to image lymph nodes as compared to the conventionally used absorbance dye lymphazurin was analyzed. When fluorescein was injected under the nipple as described above and the axillary lymph node was imaged non-invasively (without surgical exposure), results showed that both the normal and tumor-associated lymphatics had identical fluorescence spectrum for the fluorescein (FIG. 9e). However, when the same experiment was carried out after injecting 1% lymphazurin, there was a drastic difference between normal- and tumor-associated lymphatics as can be seen in FIG. 9f. This lymphazurin-induced enhanced contrast in spectral reflectance imaging clearly demonstrates an optimal strategy for detecting the physiological changes in the metastatic tumor lymph nodes by the contrast agent such as lymphazurin and a spectrally-resolved imaging platform.

Autofluorescence of the tissue stems mainly from tryptophan, collagen, elastin, NAD(P)H, flavoproteins and porphyrins. The plausible molecular source of the observed difference in spectral reflectance and fluorescence between tumor and non-tumor regions can be flavoproteins (which have emission in the 510-550 nm region). Pioneering work by Alfano et al. indicated that ratio of autofluorescence intensity at 340 nm and 440 nm could be used to distinguish cancerous and non-cancerous tissues [45]. More recent studies further point out the importance of measuring endogenous tissue fluorescence for disease diagnosis [20, 46, 47]. A major hurdle in conventional intensity imaging is that skin autofluoresence/reflectance usually obscures the optical signals that emanate from the underlying tumor. This problem stems from the fact that conventional intensity imaging relies on using emission filters (typically 60-80 nm bandwidth) that collect light over a relatively broader range of wavelengths. The approach described herein uses an AOTF, which overcame this problem by spectral separation of the signals with a narrower (˜15-20 nm bandwidth) spectral selection window. Analysis revealed that the above spectrally resolved imaging feature adds a reliable method to vascular imaging. As shown in FIG. 8e, reflectance profiles around three regions of interest shows significant differences around 460-480 nm and 600-640 nm windows thereby offering a possibility for ratiometric imaging that could potentially discriminate the skin, blood and tumor vasculature components reliably well. Although fluorescein did not yield any significant advantage over lymphazurin in enhancing spectral reflectance contrast, it has advantages in vascular imaging as exemplified in FIG. 8f.

Finally, the lymphazurin-induced enhanced contrast in spectral reflectance images of the metastatic lymph node clearly indicates that physiological tissue changes that accompany tumorigenesis/metastasis can be readily detected non-invasively without surgical complications as confirmed by similar published studies. A plausible explanation for the observed reflectance profiles in the metastatic lymph nodes is that it could arise from local changes in the vascular oxygenation and/or osmotic pressure around the lymphatics. It is a well-established fact that as the tumor size increases, oxygen partial pressure (pO2) decreases and the interstitial fluid pressure (IFP) increases [48-50]. It has been hypothesized that these changes could arise from the abnormalities in lymph vessels, leakiness in tumor vasculature as well as due to the contraction of the interstitial space mediated by stromal fibroblasts [50]. As IFP is now considered as a prognostic factor for tumor aggressiveness as well as for the efficacy of chemotherapeutic response in patients with advanced tumors, FIG. 9f points to a possibility for non-invasive monitoring of the changes in the metastatic lymph nodes thereby augmenting the current approaches for staging the tumors and monitoring chemotherapy response.

Applicant has demonstrated a viable imaging platform for real-time monitoring of tumors in preclinical rat models of breast cancer where tumor-specific spectral signatures could be imaged non-invasively with an AOTF. Early detection of tumors is the key to effective therapeutic intervention and successful patient survival. Results demonstrate an attractive strategy that can augment the existing clinical imaging repertoire with added advantages of higher spatial resolution and non-ionizing radiation. Since the contrast agents (lymphazurin and fluorescein) employed in this study are already in clinical use, these studies may be extended in a clinical setting with appropriate imaging system adaptations. AOTF, owing to its high speed acquisition, can provide a useful multimodality platform in conjunction with fast fluorescence lifetime imaging system thereby increasing sensitivity and accuracy in tumor imaging applications [51, 52].

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

### REFERENCES

1. Destounis, S., Hanson, S., Morgan, R., Murphy, P., Somerville, P., Seifert, P., Andolina, V., Arieno, A., Skolny, M. and Logan-Young, W. (2009) Computer-aided detection of breast carcinoma in standard mammographic projections with digital mammography. Int J Comput Assist Radio! Surg, 4, 331-6.

2. Brem, R. F., Ioffe, M., Rapelyea, J. A., Yost, K. G., Weigert, J. M., Bertrand, M. L. and Stern, L. H. (2009) Invasive lobular carcinoma: detection with mammography, sonography, MRI, and breast-specific gamma imaging. AJR Am J Roentgeno!, 192, 379-83.

3. Brem, R. F., Fishman, M. and Rapelyea, J. A. (2007) Detection of ductal carcinoma in situ with mammography, breast specific gamma imaging, and magnetic resonance imaging: a comparative study. Acad Radio!, 14, 945-50.

4. Pelosi, E., Arena, V., Baudino, B., Bello, M., Giani, R., Lauro, D., Ala, A., Bussone, R. and Bisi, G. (2002) Sentinel node detection in breast carcinoma. Tumori, 88, S10-1.

5. Newcomer, L. M., Newcomb, P. A., Trentham-Dietz, A., Storer, B. E., Yasui, Y., Daling, J. R. and Potter, J. D. (2002) Detection method and breast carcinoma histology. Cancer, 95, 470-7.

6. Moscinski, L. C., Trudeau, W. L., Fields, K. K. and Elfenbein, G. J. (1996) High-sensitivity detection of minimal residual breast carcinoma using the polymerase chain reaction and primers for cytokeratin 19. Diagn Mo! Patho!, 5, 173-80.

7. Jatoi, I. (1996) Detection and treatment of ductal carcinoma in situ of the breast. Jama, 276, 870-1.

8. Perdue, P., Page, D., Nellestein, M., Salem, C., Galbo, C. and Ghosh, B. (1992) Early detection of breast carcinoma: a comparison of palpable and nonpalpable lesions. Surgery, 111, 656-9.

9. Turner, D. A., Alcorn, F. S., Shorey, W. D., Stelling, C. B., Mategrano, V. C., Merten, C. W., Silver, B., Economou, S. G., Straus, A. K., Witt, T. R. and et al. (1988) Carcinoma of the breast: detection with MR imaging versus xeromammography. Radio!ogy, 168, 49-58.

10. Perhavec, A., Besic, N., Hocevar, M. and Zgajnar, J. (2008) Touch imprint cytology of the sentinel lymph nodes might not be indicated in early breast cancer patients with ultrasonically uninvolved axillary lymph nodes. Ann Surg Onco!, 15, 2257-62.

11. Guven, H. E., Bulak, H., Turanli, S. and Oral, S. (2007) Clinical importance of preoperative detection of the apical lymph node metastasis in patients with breast carcinoma. Singapore Med J, 48, 31-3.

12. Bakhshandeh, M., Tutuncuoglu, S. O., Fischer, G. and Masood, S. (2007) Use of imprint cytology for assessment of surgical margins in lumpectomy specimens of breast cancer patients. Diagn Cytopatho!, 35, 656-9.

13. Mendez, J. E., Lamorte, W. W., de Las Morenas, A., Cerda, S., Pistey, R., King, T., Kavanah, M., Hirsch, E. and Stone, M. D. (2006) Influence of breast cancer margin assessment method on the rates of positive margins and residual carcinoma. Am J Surg, 192, 538-40.

14. Mortier, M., Villeirs, G. and Kunnen, M. (1997) Lesion detection in breast carcinoma. J Be!ge Radio!, 80, 85-8.

15. Limberis, V., Romanidis, C., Galazios, G., Koutsougeras, G., Papadopoulos, N., Lambropoulou, M. and Simopoulos, C. (2009) Intraoperative estimation of sentinel lymph nodes in breast cancer by imprint cytology. Eur J Gynaeco! Onco!, 30, 85-7.

16. Zgajnar, J., Frkovic-Grazio, S., Besic, N., Hocevar, M., Vidergar-Kralj, B., Gerljevic, A. and Pogacnik, A. (2004) Low sensitivity of the touch imprint cytology of the sentinel lymph node in breast cancer patients—results of a large series. J Surg Onco!, 85, 82-6; discussion 87.

17. Quill, D. S., Leahy, A. L., Lawler, R. G. and Finney, R. D. (1984) Lymph node imprint cytology for the rapid assessment of axillary node metastases in breast cancer. Br J Surg, 71, 454-5.

18. Ramanujan, V. K., Jo, J. A., Cantu, G. and Herman, B. A. (2008) Spatially resolved fluorescence lifetime mapping of enzyme kinetics in living cells. J Microsc, 230, 329-38.

19. Ramanujan, V. K. and Herman, B. A. (2008) Nonlinear scaling analysis of glucose metabolism in normal and cancer cells. J Biomed Opt, 13, 031219.

20. Ramanujan, V. K., Zhang, J. H., Biener, E. and Herman, B. (2005) Multiphoton fluorescence lifetime contrast in deep tissue imaging: prospects in redox imaging and disease diagnosis. J Biomed Opt, 10, 051407.

21. Xiao-Jing, Z., Wan-Rong, S. and Zheng-Hui, Z. (2005) A new algorithm for watershed segmentation of cells in marrow. Conf Proc IEEE Eng Med Bio! Soc, 6, 6456-9.

22. Letteboer, M. M., Olsen, O. F., Dam, E. B., Willems, P. W., Viergever, M. A. and Niessen, W. J. (2004) Segmentation of tumors in magnetic resonance brain images using an interactive multiscale watershed algorithm. Acad Radio!, 11, 1125-38.

23. Lin, G., Adiga, U., Olson, K., Guzowski, J. F., Barnes, C. A. and Roysam, B. (2003) A hybrid 3D watershed algorithm incorporating gradient cues and object models for automatic segmentation of nuclei in confocal image stacks. Cytometry A, 56, 23-36.

24. Li, T. G., Wang, S. P. and Zhao, N. (2009) Gray-scale edge detection for gastric tumor pathologic cell images by morphological analysis. Comput Bio! Med, 39, 947-52.

25. Geback, T. and Koumoutsakos, P. (2009) Edge detection in microscopy images using curvelets. BMC Bioinformatics, 10, 75.

26. Moon, H., Chellappa, R. and Rosenfeld, A. (2002) Optimal edge-based shape detection. IEEE Trans Image Process, 11, 1209-27.

27. Trahanias, P. E. and Venetsanopoulos, A. N. (1993) Color edge detection using vector order statistics. IEEE Trans Image Process, 2, 259-64.

28. Smith, T. G., Jr., Marks, W. B., Lange, G. D., Sheriff, W. H., Jr. and Neale, E. A. (1988) Edge detection in images using Marr-Hildreth filtering techniques. J Neurosci Methods, 26, 75-81.

29. Kikuchi, A., Kozuma, S., Yasugi, T. and Taketani, Y. (2006) 3-D fractal tumor growth of epithelial ovarian cancer. Eur J Gynaeco! Onco!, 27, 561-5.

30. Dey, P. (2005) Basic principles and applications of fractal geometry in pathology: a review. Ana! Quant Cyto! Histo!, 27, 284-90.

31. Gazit, Y., Baish, J. W., Safabakhsh, N., Leunig, M., Baxter, L. T. and Jain, R. K. (1997) Fractal characteristics of tumor vascular architecture during tumor growth and regression. Microcircu!ation, 4, 395-402.

32. Vilela, M. J., Martins, M. L. and Boschetti, S. R. (1995) Fractal patterns for cells in culture. J Patho!, 177, 103-7.

33. Yeap, B. H., Muniandy, S., Lee, S. K., Sabaratnam, S. and Singh, M. (2007) Specimen shrinkage and its influence on margin assessment in breast cancer. Asian J Surg, 30, 183-7.

34. Graham, R. A., Homer, M. J., Katz, J., Rothschild, J., Safaii, H. and Supran, S. (2002) The pancake phenomenon contributes to the inaccuracy of margin assessment in patients with breast cancer. Am J Surg, 184, 89-93.

35. Motomura, K., Nagumo, S., Komoike, Y., Koyama, H. and Inaji, H. (2008) Accuracy of imprint cytology for intraoperative diagnosis of sentinel node metastases in breast cancer. Ann Surg, 247, 839-42.

36. Rovera F, Frattini F, Marelli M, Corben A D, Dionigi G, Boni L, Dionigi R: Axillary sentinel lymph node biopsy: an overview. International journal of surgery (London, England) 2008, 6 Suppl 1:S109-112.

37. Keshtgar M, Hamidian Jahromi A, Davidson T, Escobar P, Mallucci P, Mosahebi A, Baum M: Tissue screening after breast reduction. BMJ (Clinical research ed 2009, 338 :b630.

38. Keshtgar M R, Chicken D W, Tobias J S: New approaches in breast cancer management: sentinel node biopsy and intraoperative radiotherapy. International journal of fertility and women's medicine 2005, 50(5 Pt 1):218-226.

39. Nyirenda N, Farkas D L, Ramanujan V K: Preclinical evaluation of nuclear morphometry and tissue topology for breast carcinoma detection and margin assessment. Breast cancer research and treatment.

40. Esposito A, Schlachter S, Schierle G S, Elder A D, Diaspro A, Wouters F S, Kaminski C F, Iliev A I: Quantitative fluorescence microscopy techniques. Methods in molecular biology (Clifton, N.J. 2009, 586:117-142.

41. Shehada R E, Marmarelis V Z, Mansour H N, Grundfest W S: Laser induced fluorescence attenuation spectroscopy: detection of hypoxia. IEEE transactions on bio-medical engineering 2000, 47(3):301-312.

42. Svanberg K, of Klinteberg C, Nilsson A, Wang I, Andersson-Engels S, Svanberg S: Laser-based spectroscopic methods in tissue characterization. Annals of the New York Academy of Sciences 1998, 838:123-129.

43. Shonat R D, Wachman E S, Niu W, Koretsky A P, Farkas D L: Near-simultaneous hemoglobin saturation and oxygen tension maps in mouse brain using an AOTF microscope. Biophysical journal 1997, 73(3): 1223-1231.

44. Wachman E S, Niu W, Farkas D L: AOTF microscope for imaging with increased speed and spectral versatility. Biophysical journal 1997, 73(3):1215-1222.

45. Alfano R R, Das B B, Cleary J, Prudente R, Celmer E J: Light sheds light on cancer—distinguishing malignant tumors from benign tissues and tumors. Bulletin of the New York Academy of Medicine 1991, 67(2) :143-150.

46. Andersson-Engels S, Klinteberg C, Svanberg K, Svanberg S: In vivo fluorescence imaging for tissue diagnostics. Physics in medicine and biology 1997, 42(5):815-824.

47. Keller M D, Majumder S K, Kelley M C, Meszoely I M, Boulos F I, Olivares G M, Mahadevan-Jansen A: Autofluorescence and diffuse reflectance spectroscopy and spectral imaging for breast surgical margin analysis. Lasers in surgery and medicine, 42(1):15-23.

48. Brown J Q, Wilke L G, Geradts J, Kennedy S A, Palmer G M, Ramanujam N: Quantitative optical spectroscopy: a robust tool for direct measurement of breast cancer vascular oxygenation and total hemoglobin content in vivo. Cancer research 2009, 69(7):2919-2926.

49. Bird B, Bedrossian K, layer N, Miljkovic M, Romeo M J, Diem M: Detection of breast micro-metastases in axillary lymph nodes by infrared micro-spectral imaging. The Analyst 2009, 134(6):1067-1076.

50. Heldin C H, Rubin K, Pietras K, Ostman A: High interstitial fluid pressure—an obstacle in cancer therapy. Nature reviews 2004, 4(10):806-813.

51. Jo J A, Fang Q, Marcu L: Ultrafast Method for the Analysis of Fluorescence Lifetime

Imaging Microscopy Data Based on the Laguerre Expansion Technique. IEEE journal of quantum electronics 2005, 11(4):835-845.

52. Shrestha S, Applegate B E, Park J, Xiao X, Pande P, Jo J A: High-speed multispectral fluorescence lifetime imaging implementation for in vivo applications. Optics letters, 35(15):2558-2560.

What is claimed is:

1. A method for detecting a tumor margin in a subject in need thereof, comprising:
(i) providing a tissue sample from the subject;
(ii) measuring nuclear morphometric and/or tissue topology parameters in the tissue sample to discriminate between normal and tumor tissue; and
(iii) identifying a tumor margin in the tissue sample.

2. The method of claim 1, wherein the method is performed intra-operatively.

3. The method of claim 1, wherein the method is performed in vivo, in vitro or ex vivo.

4. The method of claim 1, wherein the tissue sample comprises sentinel lymph node and/or cancerous tissue of a type selected from the group consisting of breast cancer, colon cancer, lung cancer, hepatocellular cancer, gastric cancer, pancreatic cancer, cervical cancer, ovarian cancer, liver cancer, bladder cancer, cancer of the urinary tract, thyroid cancer, renal cancer, carcinoma, melanoma, head and neck cancer, brain cancer and prostate cancer.

5. The method of claim 1, wherein the nuclear morphometric parameters are nuclear size, nuclear circularity, nuclear count and/or nuclear area fraction.

6. The method of claim 5, wherein the nuclear area fraction is a function of nuclear size and nuclear count.

7. The method of claim 5, wherein a higher nuclear count and/or nuclear size in the tissue sample relative to the surrounding tissue is indicative of a tumor region.

8. The method of claim 5, wherein a higher nuclear area fraction in the tissue sample relative to the surrounding tissue is indicative of a tumor region.

9. The method of claim 1, wherein the tissue topology parameters are complexity and/or fractal dimension.

10. The method of claim 1, wherein measuring nuclear morphometric and/or tissue topology parameters comprises using a fluorescence intensity imaging system.

11. A method for detecting a tumor in a subject in need thereof comprising:
(i) obtaining multispectral reflectance images at various wavelengths of an area of interest and a surrounding area in the subject to obtain reflectance spectra/reflectance signal of the area of interest and reflectance spectra/reflectance signal of the surrounding area; and
(ii) comparing the reflectance spectra/reflectance signal of the area of interest to the reflectance spectra/reflectance signal of the surrounding area, wherein a difference between the reflectance spectra/reflectance signal of the area of interest and the reflectance spectra/reflectance signal of the surrounding area is indicative of the presence of a tumor.

12. The method of claim 11, wherein the multispectral reflectance images are obtained using acousto-optic tunable filter (AOTF) or fiber optic probes.

13. The method of claim 11, further comprising a contrasting agent.

14. The method of claim 13, wherein the contrasting agent is a fluorescence dye selected from the group consisiting of lymphazurin and fluorescein.

15. The method of claim 11, wherein the area of interest comprises sentinel lymph node and/or cancerous tissue of a type selected from the group consisting of breast cancer, colon cancer, lung cancer, hepatocellular cancer, gastric cancer, pancreatic cancer, cervical cancer, ovarian cancer, liver cancer, bladder cancer, cancer of the urinary tract, thyroid cancer, renal cancer, carcinoma, melanoma, head and neck cancer, brain cancer and prostate cancer.

16. The method of claim 11, wherein the multispectral reflectance images are obtained in vivo, in vitro or ex vivo.

17. The method of claim 1 or 11, wherein the subject is selected from the group consisting of human, monkey, ape, dog, cat, cow, horse, goat, pig, rabbit, mouse and rat.

18. An apparatus to support a tissue sample during data acquisition, comprising:
(i) a scaffold configured to enclose the tissue sample; and
(ii) a mechanism to support the scaffold, adapted to position the tissue sample for optical analysis.

19. The apparatus of claim 18, wherein the scaffold is optically transparent.

20. The apparatus of claim 18, wherein the tissue sample size is about 1-5 cc, 5-10 cc, 10-15 cc, 15-20 cc, 20-25 cc, 25-30 cc, 30-35 cc, 35-40 cc, 40-45 cc, 45-50 cc, 50-55 cc, 55-60 cc, 60-65 cc, 65-70 cc, 70-75 cc, 75-80 cc, 80-85 cc, 85-90 cc, 90-95 cc or about 95-100 cc.

21. The apparatus of claim 18, wherein the scaffold size is adjustable and is at least 1 cm larger than the tissue sample.
